(19) United States
(12) Patent Application Publication
(10) Pub. No.: US 2008/0138290 A1
(43) Pub. Date: Jun. 12, 2008

(54) Fluoro-Carbon Emulsion Enhancement Agent for High Intensity Focused Ultrasound Treatment and Use thereof

(75) Inventors: Zhibiao Wang, Chongqing (CN); Faqi Li, Chongqing (CN); Ziwen Xiao, Chongqing (CN); Yanbing Xiao, Chongqing (CN); Liping Liu, Chongqing (CN); Zhilong Wang, Chongqing (CN)

Correspondence Address: THE WEBB LAW FIRM, P.C., 700 KOPPERS BUILDING, 436 SEVENTH AVENUE, PITTSBURGH, PA, 15219, US

(73) Assignee: CHONGQING HAIFU (HIFU) TECHNOLOGY CO., LTD., Chingqing (CN)

(21) Appl. No.: 11/794,932

(22) PCT Filed: Sep. 2, 2005
(86) PCT No.: PCT/CN05/01391

§ 371 Date: Feb. 15, 2008

(30) Foreign Application Priority Data
Jan. 10, 2005 (CN) 200510000343.9

(51) Int. Cl. A61K 49/22

(52) U.S. Cl. 424/9.5

(57) ABSTRACT

The present invention discloses a fluorocarbon emulsion enhancement agent for HIFU treatment, which can increase acoustic energy deposition at the target location during HIFU treatment. The enhancement agent comprises a discontinuous phase comprising of a core material encapsulated by a membrane-forming material and a continuous phase comprised of aqueous medium; the discontinuous phase is uniformly dispersed in the continuous phase and the particle size of the discontinuous phase ranges from 0.1-2 μm; the amount of the membrane-forming material in the enhancement agent is 0.1-100 g/L; the core material is comprised of a liquid that undergoes a liquid-gas phase transition at 38-100° C., and the amount of the core material in the enhancement agent is 5-200 ml/L. The fluorocarbon emulsion enhancement agent for HIFU treatment of the present invention can significantly change the acoustic environment of the target location and can increase acoustic energy deposition at the target location during HIFU treatment.

### FIELD OF THE PRESENT INVENTION

The present invention is related to the fields of medicine and medical treatment, specifically to the field of ultrasound treatment, and more particularly to a fluoro-carbon emulsion enhancement agent for HIFU treatment, which can increase acoustic energy deposition at the target location during HIFU treatment, and use thereof.

### BACKGROUND OF THE PRESENT INVENTION

High-intensity focused ultrasound (HIFU) as a new technique to treat tumors and other diseases has already been recognized in clinical applications. HIFU employs focused ultrasound, which provides continuous, high-intensity ultrasound energy at the focus, resulting in instantaneous thermal effects (65-100° C. ), cavitation effects, mechanical effects and sonochemical effects, to selectively cause coagulative necrosis at the focus, and prevent tumors from proliferation, invasion and metastasis.

It was demonstrated that the acoustic energy was attenuated exponentially as the transmission distance increased during the ultrasound transmission within the body (Baoqin Liu et al., Chinese Journal of Ultrasound in Medicine, 2002, 18(8):565-568). In addition, the energy during ultrasound transmission in soft tissues was attenuated due to tissue absorption, scattering, refraction, diffraction and the like, among which tissue absorption and scattering are mainly responsible for the energy loss (Ruo Feng and Zhibiao Wang as editors in chief, Practical Ultrasound Therapeutics, Science and Technology Reference Publisher of China, Beijing, 2002.14). Therefore, when the HIFU treatment is used to treat deep-seated and large-sized tumors, the acoustic energy transmitted to the target would be relatively low. Thus, therapeutic efficiency would decrease and the treatment time would be prolonged due to the acoustic energy attenuation.

Of course, although the transmitting power of the therapeutic transducer might be increased in order to improve the therapeutic efficiency, the normal tissue along the pathway of the ultrasound transmission is more likely to be burned in a high-intensity ultrasound environment.

In addition, at present, when the HIFU technique is clinically applied to a hepatic tumor that is blocked by the ribs in the pathway of the ultrasound transmission, the ribs are usually removed in order to increase the energy deposition at the target location, shorten the treatment time and improve therapeutic effects. Thus the noninvasiveness of HIFU treatment cannot be ensured, which is undesirable for the patients and doctors.

The above problems have disadvantageously limited the use of the HIFU treatment as a technique for clinical practice. Therefore, the technical problems with respect to increasing the energy deposition at target location, effectively treating the deep-seated tumors without damaging the surrounding normal tissue in the acoustic pathway, and treating a hepatic tumor that is blocked by the ribs without removal of the ribs, need to be solved urgently.

### SUMMARY OF THE INVENTION

One objective of the present invention is to provide a fluoro-carbon emulsion enhancement agent for HIFU treatment, which can enhance acoustic energy deposition at the target tissue during HIFU treatment.

Another objective of the present invention is to provide a method for enhancing acoustic energy deposition at the target location during HIFU treatment using the fluoro-carbon emulsion enhancement agent of the present invention for HIFU treatment.

A further objective of the present invention is to provide use of a fluoro-carbon emulsion enhancement agent for HIFU treatment to enhance the effectiveness of HIFU treatment.

In order to achieve the above objectives, in one embodiment, the present invention provides an enhancement agent for HIFU treatment. The enhancement agent of the present invention is a substance that can enhance acoustic energy absorption at the target location to be treated with HIFU after its administration to a biological body, i.e. a substance that can be used to reduce the acoustic energy needed to cause lesions of a target tissue (tumor and non-tumor tissue) per unit volume of the tissue during HIFU treatment. In the present invention, the types of the substances used as the enhancement agents for HIFU treatment are not particularly limited, as long as the substances are fluorocarbon emulsions and can change the acoustic environment of the target tissue and promote therapeutic acoustic energy absorption and deposition at the target tissue.

As used herein, the term “lesion” refers to the substantial change in the physiological state of a tumor or normal tissue, generally refers to the coagulative necrosis of a tumor or normal tissue. Energy efficiency factor (EEF) can be used to quantify the acoustic energy needed to cause lesions of a target tissue per unit volume of the tissue. EEF is presented by the expression of EEF=ηPt/V (unit: J/mm³), and refers to the acoustic energy needed to cause lesions of a tumor or normal tissue per unit volume of the tissue, wherein, η refers to the focusing coefficient of a HIFU transducer, which reflects the ultrasound energy focusing capacity of the transducer, here η=0.7; P refers to the total acoustic power of a HIFU source (unit: W), t refers to the total time of HIFU treatment (unit: s); and V refers to the volume of HIFU-induced lesions (unit: mm³). A substance that greatly decreases the EEF of the target tissue after its administration is more suitable to be used as the enhancement agent for HIFU treatment according to the present invention.

The enhancement agent for HIFU treatment greatly decreases the EEF of the target tissue after its administration. As a result, the ratio between the EEF of the target tissue measured before the administration of the enhancement agent (i.e. EEF_{(base)}) and the EEF of the target tissue measured after the administration of the enhancement agent (i.e. EEF₍ₘₑₐₛᵤᵣₑₘₑₙₜ₎) is more than 1, preferably more than 2, and more preferably over 4. The upper limit of the ratio is not particularly limited and a higher ratio is preferred.

Specifically, the enhancement agent for HIFU treatment of the present invention comprises a discontinuous phase comprised of a core encapsulated by a membrane-forming material and a continuous phase comprising of aqueous medium. The discontinuous phase is uniformly dispersed in the continuous phase and the particle size of the discontinuous phase ranges from 0.1-2 μm, preferably 0.1-1 μm and more preferably 0.1-0.5 μm; the amount of the membrane-forming material in the enhancement agent is 0.1-100 g/L, preferably 1-50 g/L and more preferably 5-20 g/L; the core is comprised of a liquid that undergoes a liquid-gas phase transition at 38-100° C. (namely, the liquid can turn into gas within an animal body or human body during HIFU treatment), and the amount of the core material in the enhancement agent is 5-200 ml/L, preferably 10-100 ml/L, and more preferably 20-80 ml/L.

In the above embodiment of the present invention, the membrane-forming material includes: lipids, such as 3-sn-phosphatidylcholine, 1,2-dipalmitoyl-sn-glycero-3-phosphatidylglycerol sodium salt, 1,2-distearoyl-sn-glycero-3-phosphatidylcholine, sodium 1,2-dipalmitoyl-sn-glycero-3-phosphatidate, 1,2-dipalmitoyl-sn-glycero-3-phosphatidylcholine, phosphatidylserine and hydrogenated phosphatidylserine, cholesterol, and glycolipide; saccharides, including, for example, glucose, fructose, sucrose, starch and the degradation products thereof; proteins, such as albumin, globulin, fibrinogen, fibrin, hemoglobin, and the degradation products of plant proteins and the like.

The membrane-forming material of the fluoro-carbon emulsion enhancement agent for HIFU treatment according to the present invention is preferably a biocompatible and degradable biomaterial, such as a lipid, such that the enhancement agent can be injected intravenously, transported through the blood circulation smoothly, and then phagocytosed quickly by the tissues of the human body, which are full of reticuloendothelial cells. Therefore, a mass of enhancement agent can be deposited in the tissues of the human body in a certain time, significantly changing the acoustic environment of the target tissue. Thus, the ultrasound absorption capacity of the tissue can be significantly enhanced, the acoustic energy deposition at the target tissue during HIFU treatment can be increased, and eventually the effectiveness with which clinical HIFU treatment can ablate tumor cells will be improved greatly.

In the above embodiment of the present invention, the liquid that undergoes a liquid-gas phase transition at 38-100° C. includes C₅-C₆ alkanes, such as n-pentane, and i-pentane, and C₅-C₁₂ fluorohydrocarbons , and the like (see pages 65-70 of Chinese Patent No. ZL94191564 (Application No. CN1068230C)).

In the above embodiment of the present invention, the aqueous medium is distilled water, physiological saline or glucose solution. The concentration of the glucose solution can be up to 50% (w/v). But the glucose solution cannot be used as the aqueous medium for the fluoro-carbon emulsion enhancement agent for HIFU treatment in diabetic patients.

In a preferred embodiment, the enhancement agent may contain an emulsifier. The emulsifier is typically selected from a group consisting of ethylene glycol mono-C₁₆₋₁₈-fatty acid esters, diethylene glycol mono-C₁₆₋₁₈-fatty acid esters, diethylene glycol di-C₁₆₋₁₈-fatty acid esters, triethylene glycol mono-C₁₆₋₁₈-fatty acid esters, sorbitan fatty acid ester (Span type) emulsifiers, polysorbate (Tween type) emulsifiers, polyethylene glycol monolaurate-based emulsifiers, polyoxyethylene laurate-based emulsifiers, 3-sn-phosphatidylcholine (lecithin), cholic acid, and the like. The amount of the emulsifier in the enhancement agent is 5-150 g/L. In addition, the enhancement agent may also contain a stabilizing agent, such as carboxymethylcellulose sodium (CMC-Na), carboxymethylcellulose potassium, carboxyethylcellulose sodium, carboxyethylcellulose potassium, carboxypropylcellulose sodium, carboxypropylcellulose potassium, glycerin, and the like. The amount of the CMC-Na contained in the enhancement agent is 0.01-10 g/L, preferably 0.05-0.6 g/L, and more preferably 0.1-0.3 g/L. The amount of the glycerin contained in the enhancement agent is 5-100 g/L.

In a more preferred embodiment, in order to increase the stability of the enhancement agent, the enhancement agent is adjusted to pH 7.0-9.0, preferably 7.5-8.5. Inorganic or organic acids or bases may be used to adjust the pH value of the enhancement agent.

Additionally, in order to make the fluoro-carbon emulsion enhancement agent for HIFU treatment according to the present invention target a specific tumor tissue or focus, substances having specific affinity to the tumor tissue or the focus, such as a tumor-specific antibody, may be added to the enhancement agent.

The fluoro-carbon emulsion ultrasound contrast agents that are widely used in ultrasound imaging may be used as the fluoro-carbon emulsion enhancement agent for HIFU treatment of the present invention. Thus, in another aspect, the present invention provides use of fluoro-carbon emulsion ultrasound contrast agent for preparing the enhancement agent for HIFU treatment.

In another embodiment, the present invention provides a method for preparing the fluoro-carbon emulsion enhancement agent for HIFU treatment. The method comprises:

(1) weighing and mixing a membrane-forming material and a core material to obtain 0.1-100 g/L membrane-forming material and 5-200 g/L core material, adding an aqueous medium to the mixture up to a predetermined volume, and stirring the mixture to form a coarse emulsion;

(2) emulsifying the coarse emulsion prepared in step (1) in a high pressure homogenizer (see Example 19 described in Chinese patent No. CN1068230C). Preferably, the coarse emulsion is emulsified twice.

In the method for preparing the fluoro-carbon emulsion enhancement agent for HIFU treatment of the present invention, the mixture is preferably stirred and dispersed in an ice bath in the above-mentioned step (1), and more preferably, an emulsifier and/or stabilizing agent may be added to the mixture when the membrane-forming material and core material are mixed.

The present invention is further directed to a method for increasing energy deposition at the target location during HIFU treatment, wherein, the method comprises administering an effective dosage of the fluoro-carbon emulsion enhancement agent of the present invention intravenously via continuous and rapid IV instillation or bolus injection to a patient at 0-30 minutes before applying HIFU treatment to a patient. The effective dosage mentioned above varies with the type of tumor, weight of patient, location of tumor, volume of tumor and the like. However, a doctor or a pharmacist can easily determine the suitable dosage for different patients. For example, the dosage can be selected from the range of 0.005-0.1 ml/kg, preferably 0.01-0.05 ml/kg

### DETAILED DESCRIPTION OF THE INVENTION

### EXAMPLE 1

The following materials were mixed to a final volume of 1000 ml: 3% (w/v) emulsifier Pluronic F-68 (purchased from Sigma Company), 0.5% (w/v) yolk lecithin (purchased from Shanghai Chemical Reagent Company), 5% (v/v) perfluoropentane (purchased from Sigma Company), and distilled water. The mixture was incubated on ice and sheared and dispersed at 10000 rpm for 5 minutes to obtain a coarse emulsion. The coarse emulsion was emulsified in a high-pressure homogenizer at 4° C. twice. The resulting emulsion with a particle size of less than 1 μm was obtained by filtering through a 1 μm membrane filter. The final emulsion was divided and put into 15 ml vials, and then was radiated by Co₆₀ at 20 KGY for 10 hours. The emulsion had a particle concentration of 10⁹/ml and was refrigerated for storage.

### EXAMPLE 2

The following materials were mixed to a final volume of 1000 ml: 6% (w/v) emulsifier Pluronic F-68 (purchased from Sigma Company), 1% (w/v) yolk lecithin (purchased from Shanghai Chemical Reagent Company), 10% (v/v) perfluoropentane (purchased from Sigma Company), and physiological saline solution. The mixture was incubated on ice and sheared and dispersed at 10000 rpm for 5 minutes to obtain a coarse emulsion. The coarse emulsion was emulsified in a high-pressure homogenizer at 4° C. twice. The resulting emulsion with a particle size of less than 1 μm was obtained by filtering through a 1 μm membrane filter. The final emulsion was divided and put into 15 ml vials, and then was radiated by Co₆₀ at 20 KGY for 10 hours. The emulsion had a particle concentration of of 10⁹/ml and was refrigerated for storage.

### EXAMPLE 3-6

The fluoro-carbon emulsion enhancement agents for HIFU treatment of the present invention were prepared according to the same method and procedures described in Example 1 with the materials and the amounts thereof set forth in Table 1. The parameters of the products are shown in Table 1.

TABLE 1

| | Example 3 | Example 4 | Example 5 | Example 6 |
|---|---|---|---|---|
| Core material | 2% (v/v) | 5% (v/v) | 10% (v/v) | 10% (v/v) |
| | Perfluoro- | Perfluoro- | Perfluoro- | Dihydrodecafluoro- |
| | pentane | hexane | hexane | pentane |
| Lecithin | 1% (w/v) | 2% (w/v) | 2% (w/v) | 2% (w/v) |
| Glycerin | 1% (w/v) | 1% (w/v) | 1% (w/v) | 1% (w/v) |
| Pluronic F-68 | 5% (w/v) | 3% (w/v) | 5% (w/v) | 5% (w/v) |
| Final volume | 1000 ml | 1000 ml | 1000 ml | 1000 ml |
| after distilled | | | | |
| water added | | | | |
| PH (c.a.) | 6.98 | 7.01 | 6.99 | 7.00 |
| Particle size of | 0.5-2 μm | 0.5-2 μm | 0.1-2 μm | 1-2 μm |
| the | | | | |
| discontinuous | | | | |
| phase | | | | |

The effects of the enhancement agent for HIFU treatment of the present invention are demonstrated below through the combined use of the fluoro-carbon emulsion enhancement agent for HIFU treatment as prepared in Example 1 and HIFU Tumor Therapeutic System Model-JC.

### Animal Test 1 Study on Lesions of New Zealand White Rabbit Liver

Twenty New Zealand white rabbits weighing 2.21±0.56 kg, which were provided by the Laboratory Animals Center of Chongqing University of Medical Sciences, were used. These rabbits were shaved at the lower bosom and the midsection on the day prior to study. A High-intensity Focused Ultrasound Tumor Therapeutic System Model-JC manufactured by Chongqing Haifu (HIFU) Technology Co. Ltd. was used to radiate these rabbits. The High-intensity Focused Ultrasound Tumor Therapeutic System Model-JC was composed of an adjustable power generator, a B-mode ultrasound monitoring system, a therapeutic transducer, a mechanical motion control system, a treatment bed and an acoustic coupling device. The therapeutic transducer of the System, with working frequency of 1 MHz, diameter of 150 mm, and focal distance of 150 mm, using standard circulating degassed water with gas content of less than or equal to 3 ppm, can produce a focal region of 2.3×2.4×26 mm and deliver an average acoustic intensity of 5500 W/cm². The transducer used in this study was 150 mm in diameter, and it had a focal distance of 135 mm, a working frequency of 1.0 MHz and an acoustic power of 200 W. The exposure depth was 20 mm, and the discontinuous single pulse exposure with exposure duration of 3 s and interval of 5 s was applied. The physiological saline solution (0.02 ml/kg) was rapidly delivered via rabbit ear border vein to each rabbit, and the rabbit liver was exposed to HIFU using single pulse exposure 60 seconds later on the control side. The enhancement agent for HIFU treatment as prepared in Example 1 (0.02 ml/kg) was rapidly delivered via rabbit ear border vein to each rabbit, and the other plane of the same rabbit liver of the control side were exposed with HIFU at 60 seconds later for the experimental side. The ultrasound exposures finished when gray-scale changes occurred at the target location. If there is no gray-scale change to be seen, the total exposure duration should be no more than 20 s. Three days after ultrasound exposure, the rabbits were sacrificed by breaking the necks and were dissected. The volume (V) of coagulative necrosis of rabbit liver was measured. The EEF was calculated according to the expression EEF=ηPt/V, wherein, t refers to the exposure time and η=0.7. The median of the EEFs was 6.0160 on the control side and 1.2505 on the experimental side. Wilcoxon signed rank-sum test showed Z=−2.485, and P=0.013. The results of this study show that the fluorocarbon emulsion increases the effectiveness of HIFU to cause lesions of the rabbit livers. In fact, the mean of the EEF in the control side is 4.81 times as much as the mean of the EEF in the experimental side.

### Animal Test 2 Study on Lesions of Goat Liver

Twenty Nanjiang yellow goats weighing 22.25±4.51 kg were used. The goats were shaved at the right bosom and the right abdomen on the day of the study. A High-intensity Focused Ultrasound Tumor Therapeutic System Model-JC manufactured by Chongqing Haifu (HIFU) Technology Co. Ltd. was used to radiate these yellow goats. The transducer used in this study was 150 mm in diameter, and it had a focal distance of 135 mm, a working frequency of 0.8 MHz and an acoustic power of 220 W. The exposure depth was 30 mm, and a discontinuous single pulse exposure with exposure duration of 3 s and interval of 5 s was applied. Ribs of all the goats were not removed. A pre-scanning was carried out before HIFU exposure and the areas for exposure including 4 planes were selected. One exposure spot was introduced on each plane, and two-dimensional ultrasound was used to monitor the rib clearance. The physiological saline solution (0.02 ml/kg) was rapidly delivered intravenously via ear border to each goat, and the goat liver was exposed to HIFU 60 seconds later and two exposure spots were introduced on each goat on the control side. The enhancement agent for HIFU treatment as prepared in Example 1 (0.02 ml/kg) was rapidly delivered intravenously via ear border to each goat, and the goat liver was exposed to HIFU 60 seconds later and two exposure spots were introduced on each goat on the experimental side. When gray-scale changes occurred at the target location, the exposures were repeated another 4 or 5 times. If there is no gray-scale change to be seen, the total exposure duration should be no more than 200 s. Three days after ultrasound exposure, the goats were sacrificed and dissected. The volume (V) of coagulative necrosis of goat liver was measured. The EEF was calculated according to the expression EEF=ηPt/V, wherein, T refers to the exposure time and η=0.7. The median of EEFs was infinite for the control side and 5.1904 for the experimental side using the combination of HIFU and fluoro-carbon emulsion. Wilcoxon signed rank-sum test showed P=0.004. This study shows that the fluoro-carbon emulsion significantly increased the ability of HIFU to create lesions in goat livers without removal of the ribs of the goats.

### Animal Test 3 Study on Lesions of Goat Kidney

Twenty Nanjiang yellow goats weighing 22.25±4.51 kg were used. These goats were shaved at the right bosom and the right abdomen on the day of the study. A High-intensity Focused Ultrasound Tumor Therapeutic System Model-JC manufactured by Chongqing Haifu (HIFU) Technology Co. Ltd. was used to radiate these yellow goats. The transducer used in this study was 150 mm in diameter, and it had a focal distance of 135 mm, a working frequency of 0.8 MHz and an acoustic power of 220 W. The exposure depth was 20 mm, and a discontinuous single pulse exposure with exposure duration of 3 s and interval of 5 s was applied. Ribs of all the goats were not removed. A pre-scan was carried out before HIFU exposure and the areas for exposure including 1 plane on the upper pole of kidney and 1 plane on the lower pole of kidney respectively were selected. One exposure spot was introduced on each plane, and two-dimensional ultrasound was used for observation. The right ribs were avoided if they become obstacles. The physiological saline solution (0.02 ml/kg) was rapidly delivered intravenously via ear border to each goat, and the goat kidney was exposed to HIFU under single pulse exposure 30 seconds later on the control side. The enhancement agent for HIFU treatment as prepared in Example 1 (0.02 ml/kg) was delivered rapidly intravenously via ear border to each goat, and the goat kidney was exposed to HIFU 60 seconds later for the experimental side. When gray-scale changes occurred at the target location, the exposures were repeated another 3 or 4 times. If there is no gray-scale change to be seen, the total exposure duration should be no more than 150 s. Three days after ultrasound exposure, the goats were sacrificed and dissected. The volume (V) of coagulative necrosis of goat kidney was measured. The EEF was calculated according to the expression EEF=ηPt/V, wherein, T refers to the exposure time and η=0.7. The EEFs were 10.58±3.95 for the experimental side and 486.37±215.41 for the control side. Wilcoxon signed rank-sum test showed P=0.008. The results of this study indicate that the fluoro-carbon emulsion greatly increased the ability of HIFU to cause lesions in normal goat kidneys. In fact, the mean of the EEF in the control side is more than 40 times the mean of the EEF in the experimental side.

### INDUSTRIAL APPLICABILITY

The fluoro-carbon emulsion enhancement agent for HIFU treatment of the present invention can change the acoustic environment of the target location greatly and can reduce the acoustic energy needed to cause lesions of a target tissue (tumor and non-tumor tissue) per unit volume of the tissue during HIFU treatment. Accordingly, deep-seated and large-sized tumors can be treated with HIFU treatment more effectively under a certain acoustic power without damaging the normal tissue along the acoustic pathway. It becomes possible to use the enhancement agent for HIFU treatment of the present invention to effectively treat patients with hepatic tumors that are blocked by the ribs without removal of the ribs.

Although the present invention has been described in connection with the preferred embodiments, it is not intended to limit the scope of the present invention by the above descriptions of the embodiments. It should be understood that various modifications and changes to which the present invention may be applicable will be readily apparent to those skilled in the art. The claims are intended to cover the scope of the present invention.

1. An enhancement agent for high intensity focused ultrasound (HIFU) treatment, wherein the enhancement agent comprises a discontinuous phase comprised of a core material encapsulated by a membrane-forming material and a continuous phase comprised of aqueous medium, the discontinuous phase is uniformly dispersed in the continuous phase and the particle size of the discontinuous phase ranges from 0.1-2 μm, wherein the amount of the membrane-forming material in the enhancement agent is 0.1-100 g/L, and wherein the core material is comprised of a liquid that undergoes a liquid-gas phase transition at 38-100° C. and the amount of the core material in the enhancement agent is 5-200 ml/L.

2. The enhancement agent according to claim 1, wherein the discontinuous phase has a particle size ranging from 0.1-1 μm.

3. The enhancement agent according to claim 2, wherein the discontinuous phase has a particle size ranging from 0.1-0.5 μm.

4. The enhancement agent according to claim 1, wherein the membrane-forming material is one or more substances selected from the group consisting of phospholipin, cholesterol and glycolipide.

5. The enhancement agent according to claim 4, wherein the membrane-forming material comprises phospholipin selected from the group consisting of 3-sn-phosphatidylcholine, 1,2-dipalmitoyl-sn-glycero-3-phosphatidylglycerol sodium salt, 1,2-distearoyl-sn-glycero-3-phosphatidylcholine, sodium 1,2-dipalmitoyl-sn-glycero-3-phosphatidate, 1,2-dipalmitoyl-sn-glycero-3-phosphatidylcholine, phosphatidylserine and hydrogenated phosphatidylserine.

6. The enhancement agent according to claim 1, wherein the amount of the membrane-forming material in the enhancement agent is 1-50 g/L.

7. The enhancement agent according to claim 6, wherein the amount of the membrane-forming material in the enhancement agent is 5-20 g/L.

8. The enhancement agent according to claim 1, wherein the core material is one or more substances selected from the group consisting of C₅-C₆ alkanes and C₅-C₁₂ fluorohydrocarbons.

9. The enhancement agent according to claim 8, wherein the core material is selected from the group consisting of perfluoropentane and dihydrodecafluoropentane.

10. The enhancement agent according to claim 1, wherein the amount of the core material in the enhancement agent is 10-100 ml/L.

11. The enhancement agent according to claim 10, wherein the amount of the core material in the enhancement agent is 20-80 ml/L.

12. The enhancement agent according to claim 1, wherein the aqueous medium is comprised of distilled water, physiological saline solution or glucose solution.

13. The enhancement agent according to claim 1, wherein the enhancement agent contains an emulsifier in an amount of 5-150 g/L, and wherein the emulsifier is selected from the group consisting of ethylene glycol mono-C₁₆₋₁₈-fatty acid esters, diethylene glycol mono-C₁₆₋₁₈-fatty acid esters, diethylene glycol di-C₁₆₋₁₈-fatty acid esters, triethylene glycol mono-C₁₆₋₁₈-fatty acid esters, sorbitan fatty acid esters, polysorbate, polyethylene glycol monolaurate, polyoxyethylene laurate, 3-sn-phosphatidylcholine, polyethylene oxide/polypropylene oxide/ethylene glycol non-ionic block copolymer, fluorosurfactant, and cholic acid.

14. The enhancement agent according to claim 1, wherein the enhancement agent contains a stabilizing agent comprising carboxymethylcellulose sodium, and wherein the amount of the carboxymethylcellulose sodium in the enhancement agent is 0.01-10 g/L.

15. The enhancement agent according to claim 1, wherein the enhancement agent contains a stabilizing agent comprising glycerin, and wherein the amount of the glycerin in the enhancement agent is 5-100 g/L.

16. A method for increasing acoustic energy deposition at a target location during HIFU treatment, comprising the step of:
administering the enhancement agent according to claim 1 in an effective dosage intravenously via continuous and rapid IV instillation or bolus injection to a patient at 0-30 minutes before the application of HIFU treatment to the target location of a patient.

17. (canceled)

18. The enhancement agent according to claim 2, wherein the enhancement agent contains an emulsifier in an amount of 5-150 g/L, and wherein the emulsifier is selected from the group consisting of ethylene glycol mono-C₁₆₋₁₈-fatty acid esters, diethylene glycol mono-C₁₆₋₁₈-fatty acid esters, diethylene glycol di-C₁₆₋₁₈-fatty acid esters, triethylene glycol mono-C₁₆₋₁₈-fatty acid esters, sorbitan fatty acid esters, polysorbate, polyethylene glycol monolaurate, polyoxyethylene laurate, 3-sn-phosphatidylcholine, polyethylene oxide/polypropylene oxide/ethylene glycol non-ionic block copolymer, fluorosurfactant, and cholic acid.

19. The enhancement agent according to claim 2, wherein the enhancement agent contains a stabilizing agent comprising carboxymethylcellulose sodium, and wherein the amount of the carboxymethylcellulose sodium in the enhancement agent is 0.01-10 g/L.

20. The enhancement agent according to claim 2, wherein the enhancement agent contains a stabilizing agent comprising glycerin, and wherein the amount of the glycerin in the enhancement agent is 5-100 g/L.

21. A method for increasing acoustic energy deposition at a target location during HIFU treatment, comprising the step of:
administering the enhancement agent according to claim 2 in an effective dosage intravenously via continuous and rapid IV instillation or bolus injection to a patient at 0-30 minutes before the application of HIFU treatment to the target location of a patient.
